(19) United States
(12) Patent Application Publication
(10) Pub. No.: US 2011/0029089 A1
(43) Pub. Date: Feb. 3, 2011

(54) Shoulder Prosthesis

(75) Inventors: Alessio Giuliani, Pescara (IT); Valter Bocenti, Piacenza (IT); Elia Brambilla, Cassano D'Adda (IT); Giuseppe Sala, Desio (IT); Paolo Guerra, Milano (IT); Luca Rizzi, Milano (IT)

Correspondence Address: SHOEMAKER AND MATTARE, LTD, 10 POST OFFICE ROAD - SUITE 100, SILVER SPRING, MD, 20910, US

(73) Assignee: PLUSTEK S.R.L., Milano (IT)

(21) Appl. No.: 12/376,201

(22) PCT Filed: Aug. 3, 2007
(86) PCT No.: PCT/IT2007/000567

§ 371 Date: May 20, 2009

(30) Foreign Application Priority Data
Aug. 3, 2006 (IT) MI2006A001568

(51) Int. Cl. A61F 2/40

(52) U.S. Cl. 623/19.14

(57) ABSTRACT

A shoulder prosthesis of a modular type adapted to changing from a direct configuration to an inverted configuration. The prosthesis is particularly non-invasive and has a large modular capacity, permitting full recovery of the articular function following the implant thereof.

### FIELD OF THE INVENTION

The present invention relates to shoulder prosthesis and in particular to modular type shoulder prosthesis, suitable for changing from a direct configuration to an inverted configuration. The direct configuration consists of a shoulder prosthesis wherein a head is associated with the main body of the prosthesis to replace the humeral head, suitable for being coupled with the glenoid, while the inverted configuration consists of a shoulder prosthesis wherein a ball is associated with the glenoid suitable for being coupled with a cup associated with the humerus.

### BACKGROUND OF THE INVENTION

Shoulder prostheses according to prior art comprise a main prosthesis body adapted for insertion into the humerus and a ball and socket type coupling to permit relative movements between humerus and glenoid.

However, said prostheses present numerous drawbacks. For example the prostheses according to prior art do not permit complete recovery of the biomechanical articular function after surgery. In fact the loss of recovery of this function is mainly caused by incorrect suturing of the tuberosities of the humeral head. Lack of functional recovery often makes the prosthesis painful for the patient, and in any case, it limits the shoulder movement of the patient to a large extent.

Till now, therefore, current shoulder prostheses often have not provided satisfactory recovery of the articular function.

Furthermore, the prostheses according to prior art often result as being excessively invasive and therefore, in order to permit their insertion, a large amount of bone must be removed, thus causing problems for prosthesis integration and excessive weakening of the bone, especially in the area of the neck of the humeral head.

Furthermore, the prostheses according to prior art often do not permit the correction of certain important geometric parameters such as the distance between the acromion and the head and the version angle of the head, except by extremely invasive operations requiring the explant of the prosthesis and its total or partial replacement. These operations require interventions that are extremely invasive and painful for the patient, comprising the filing of the neck of the humeral head, with the consequential ablation of a considerable amount of bone.

The prostheses according to prior art do not permit safe and not particularly invasive attachment to the bone especially in relation to the glenoid. In fact it is often necessary to use numerous very invasive screws which not only complicate the prosthesis implant surgical operation considerably, but also contribute towards weakening the resistant sections of bone that are already particularly reduced from an anatomical point of view.

For anchoring to the bone, the prostheses according to prior art use pass-through screws that perforate the cortex and penetrate the spongy tissue.

The use of the drilling method complicates the surgical insertion stage also because incorrect hole drilling can lead to bone fractures. Furthermore, any error in prosthesis positioning also involves the drilling of an invasive hole which can be damaging to bone resistance.

### SUMMARY OF THE INVENTION

The object of the present invention is to provide a shoulder prosthesis that overcomes the drawbacks described in relation to prior art.

These drawbacks are overcome by shoulder prosthesis according to claim 1.

Other embodiments of the prosthesis according to the invention are described in the following claims.

### BRIEF DESCRIPTION OF THE DRAWINGS

Further characteristics and advantages of the present invention will be made more clear in the description below and the preferred non-limiting embodiments, wherein:

FIG. 1 shows a perspective view of a shoulder prosthesis according to one embodiment of the present invention;

FIG. 2 shows a front view of the prosthesis in FIG. 1;

FIG. 3 shows a cross-section view of the prosthesis in FIG. 1, along the section line III-Ill in FIG. 2;

FIG. 4 shows a side view of the prosthesis in FIG. 1;

FIG. 5 shows a front view of a detail of a prosthesis according to one embodiment of the present invention;

FIG. 6 shows a cross-section view of the detail in FIG. 5, along the section line VI-VI in FIG. 5;

FIG. 7 shows a side view of the detail in FIG. 5;

FIG. 8 shows a cross-section view of the detail in FIG. 5, along the section line VIII-VIII in FIG. 5;

FIG. 9 shows a cross-section view of the prosthesis in FIG. 3, along the section line IX-IX in FIG. 3;

FIG. 10 shows a perspective view of a shoulder prosthesis according to a further embodiment of the present invention;

FIG. 11 shows a cross-section view in relation to the section line XI-XI in FIG. 10;

FIG. 12 shows a side view of the prosthesis in FIG. 10;

FIG. 13 shows a cross-section view of a detail of the prosthesis in FIG. 10;

FIG. 14 shows a view in separate parts of a shoulder prosthesis according to a further embodiment of the present invention;

FIG. 15 shows a side view of the prosthesis in FIG. 14;

FIG. 16 shows a front view of the prosthesis in FIG. 14 in an assembled configuration;

FIG. 17 shows a side view of the prosthesis in FIG. 16;

FIGS. 18 and 19 show views from different angles of a detail of the prosthesis in FIG. 14;

FIG. 20 shows a perspective view of a prosthesis according to the invention in a configuration of the insertion into a humerus;

FIG. 21 shows a front view of a shoulder prosthesis according to a further embodiment of the present invention;

FIG. 22 shows a cross-section view of the prosthesis in FIG. 21, along the section line XXII-XXII in FIG. 21;

FIGS. 23-25 show views in separate parts of the prosthesis in FIG. 22, according to further variants of the embodiment;

FIG. 26 shows a side view of a detail of the prosthesis in FIG. 21;

FIG. 27 shows a front view of a shoulder prosthesis according to a further embodiment of the present invention;

FIG. 28 shows a cross-section view of the prosthesis in FIG. 27, along the section line XXVIII-XXVIII in FIG. 27;

FIGS. 29-31 show in separate parts of the prosthesis in FIG. 27, according to further variants of the embodiment;

FIG. 32 shows a side view of a detail of the prosthesis in FIG. 27;

FIG. 33 shows a front view of a shoulder prosthesis according to a further embodiment of the present invention;

FIG. 34 shows a cross-section view of the prosthesis in FIG. 33, along the section line XXXIV-XXXIV in FIG. 33;

FIGS. 35-37 show in separate parts of the prosthesis in FIG. 33, according to further variants of the embodiment;

FIGS. 38a and 38b show side views and in cross section of a detail of the prosthesis in FIG. 33;

FIG. 39 shows a cross-section view of an enlarged detail of a component of a prosthesis according to the present invention;

FIG. 40 shows a perspective view in an assembled configuration of a shoulder prosthesis according to a further embodiment of the present invention;

FIGS. 41 and 42 show side views from different angles of the prosthesis in FIG. 21;

FIG. 43 shows a plane view from below of the prosthesis in FIG. 40;

FIG. 44 shows a perspective view in separate parts of the prosthesis in FIG. 40;

FIG. 45 shows a perspective view of a shoulder prosthesis according to a further embodiment of the present invention;

FIGS. 46 and 47 show side views from different angles of the prosthesis in FIG. 45;

FIGS. 48A and 48B show plane views seen from above and below of the prosthesis in FIG. 45 respectively;

FIG. 49 shows a perspective view in separate parts of the prosthesis in FIG. 45;

FIG. 50 shows a perspective view of a prosthesis according to the present invention in a configuration assembled on a scapula (during preparation);

FIG. 51 shows a perspective view in separate parts of a shoulder prosthesis according to a further embodiment of the present invention;

FIG. 52 shows a perspective view in an assembled configuration of the prosthesis in FIG. 51;

FIG. 53 shows a side view of the prosthesis in FIG. 52;

FIG. 54 shows an enlarged detail of the prosthesis in FIG. 53.

FIGS. 55-58 show perspective views, side views and views in cross section of shoulder prosthesis according to a further embodiment of the present invention.

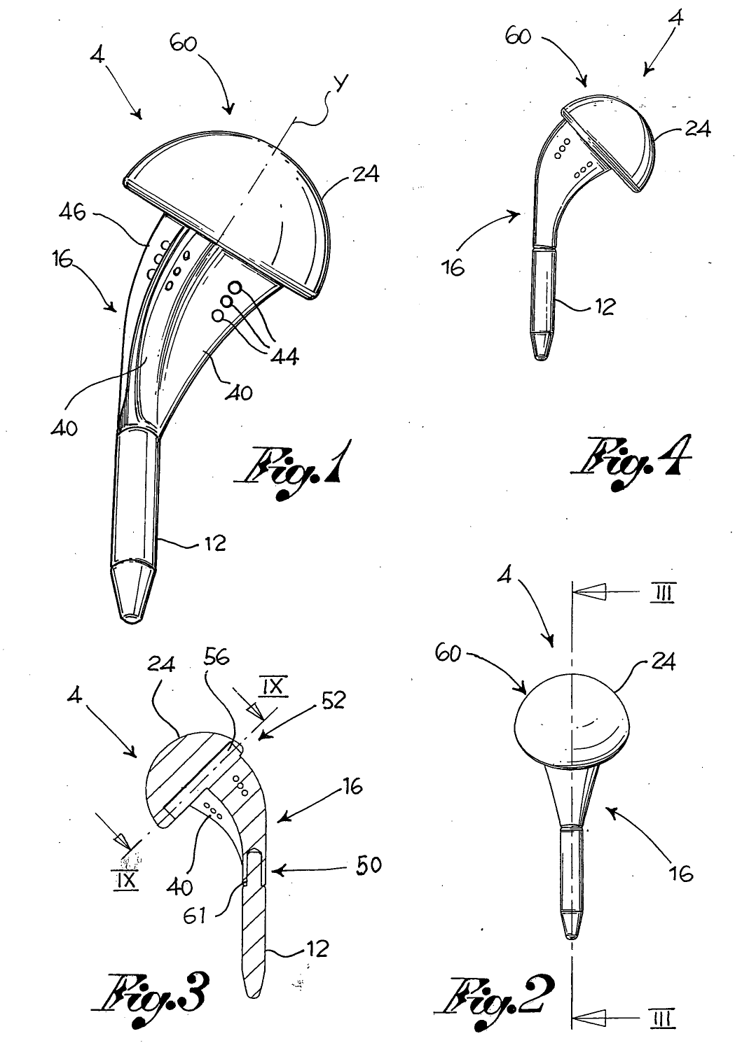

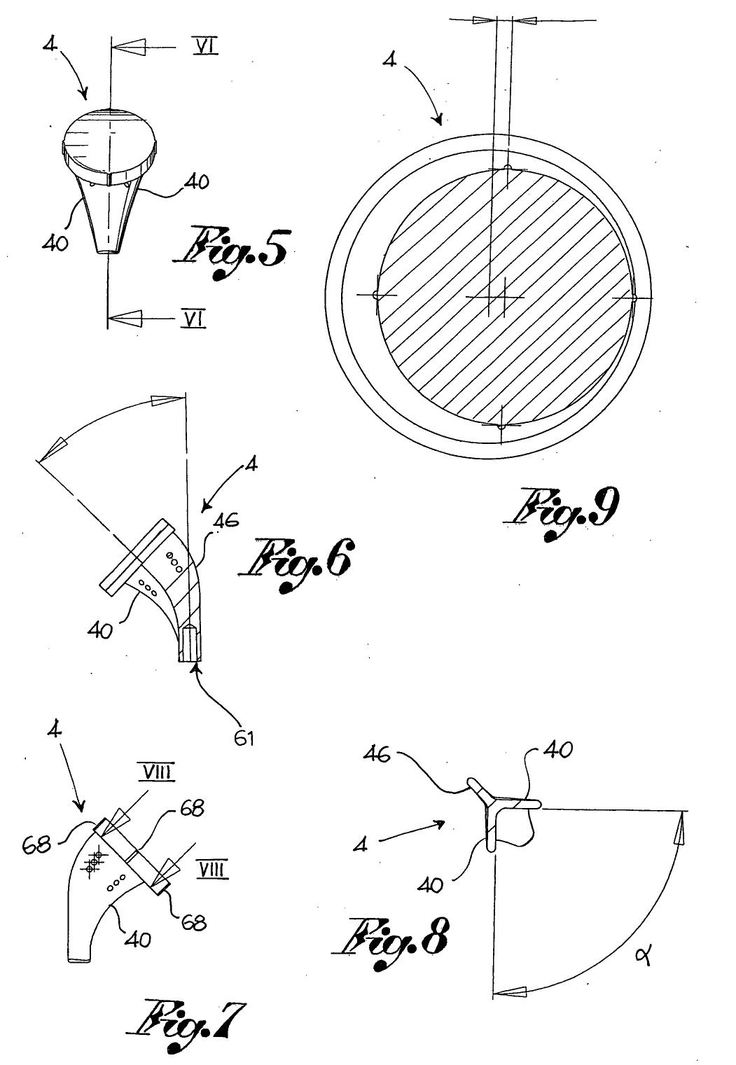

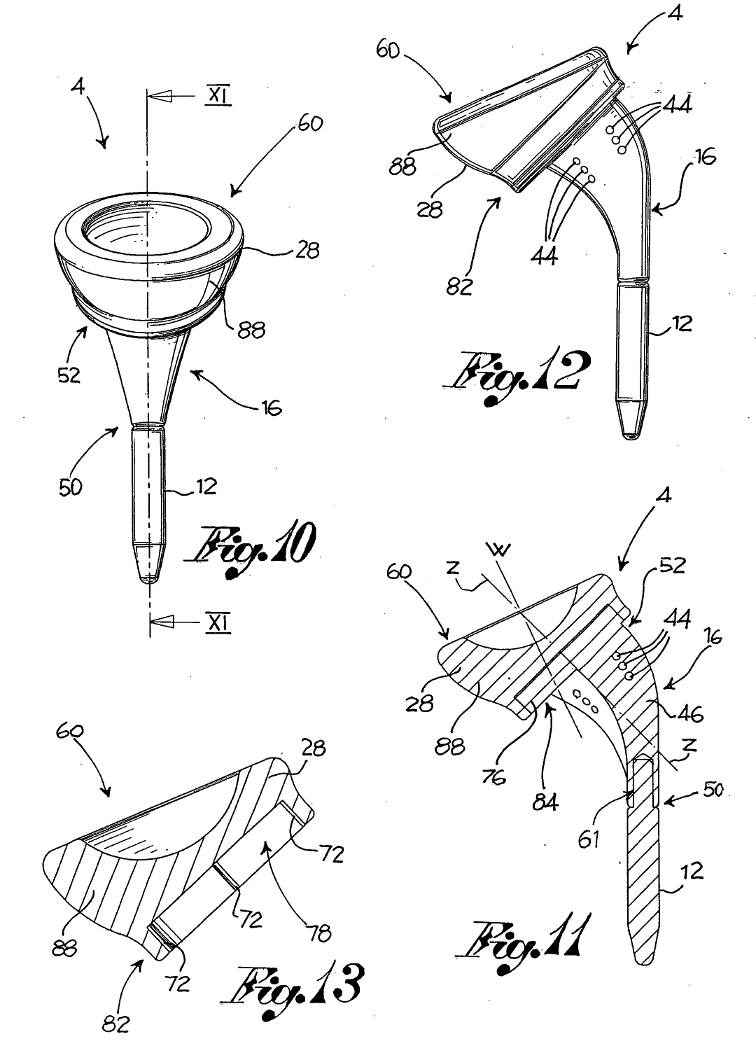

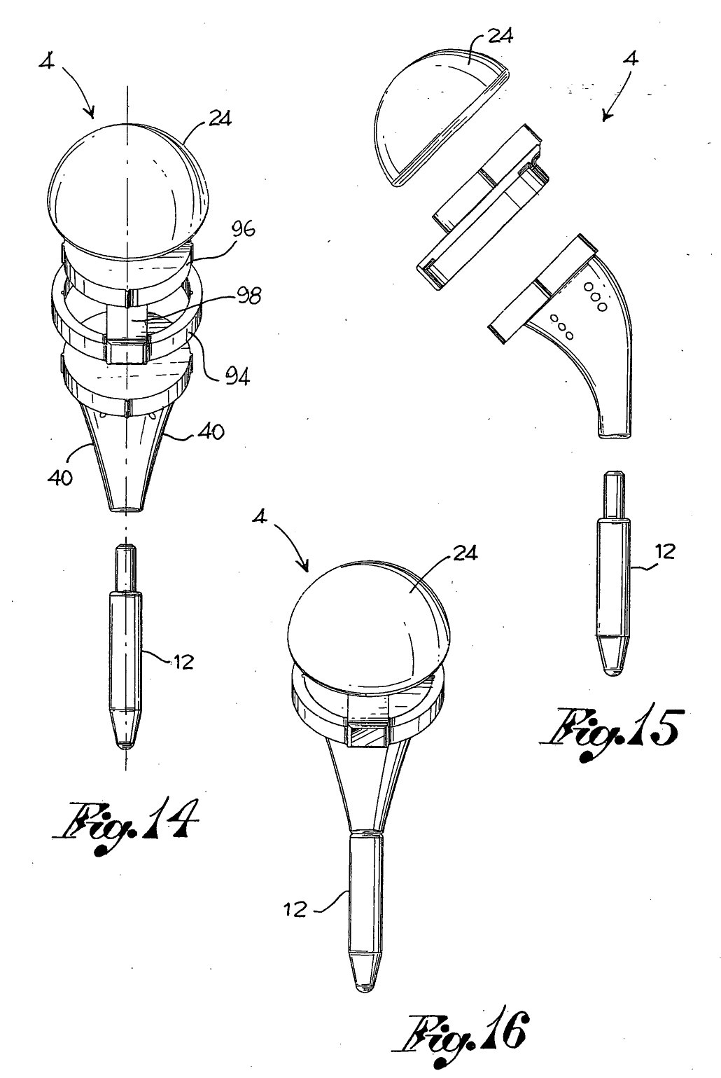

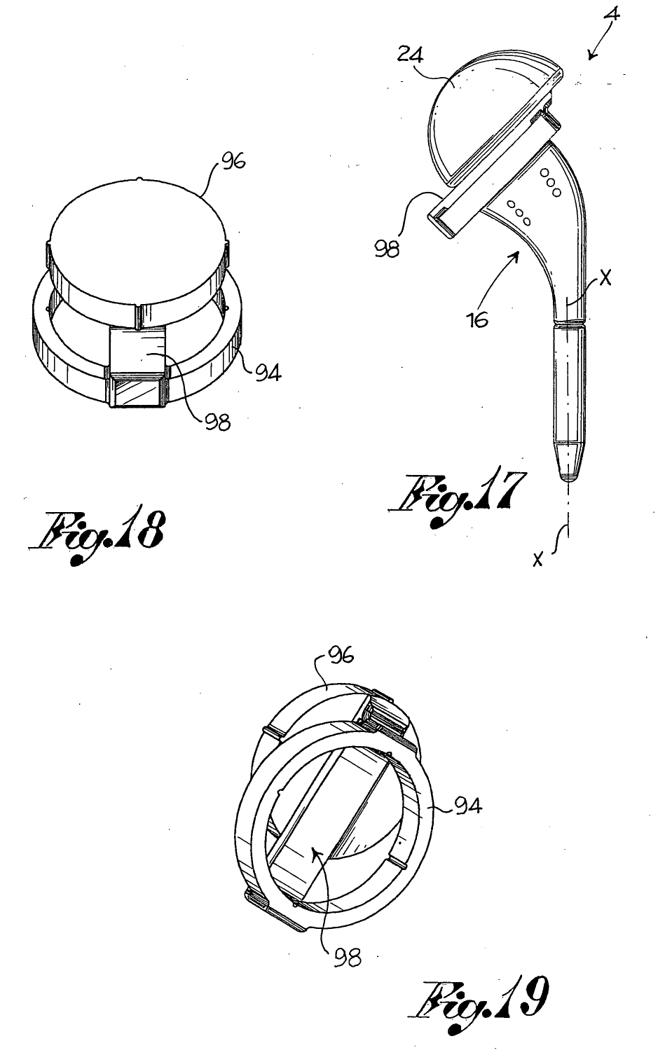

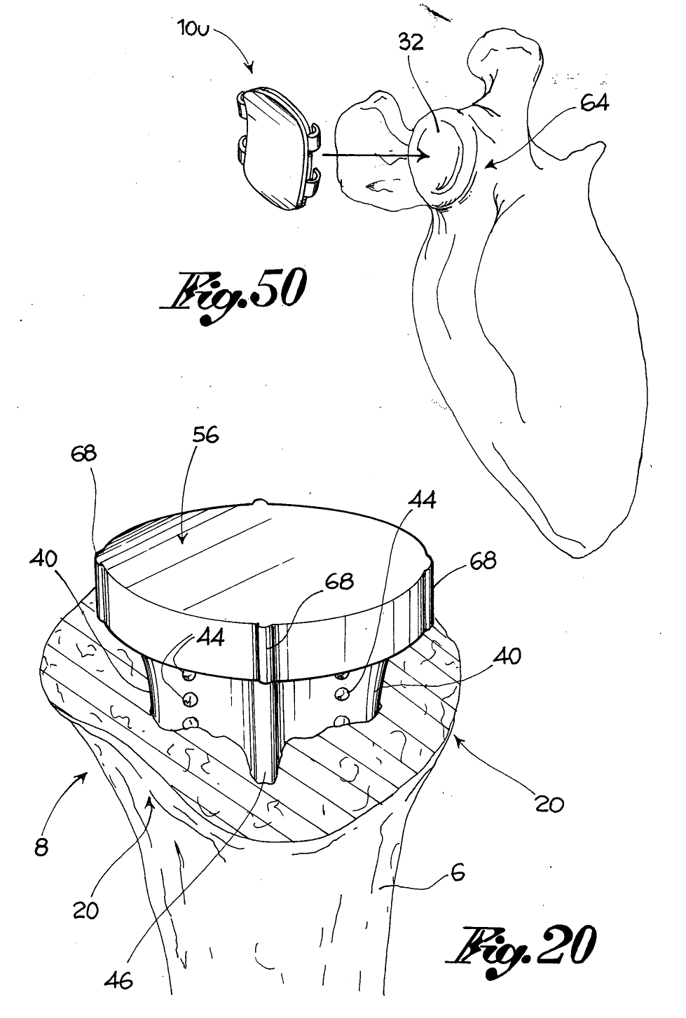

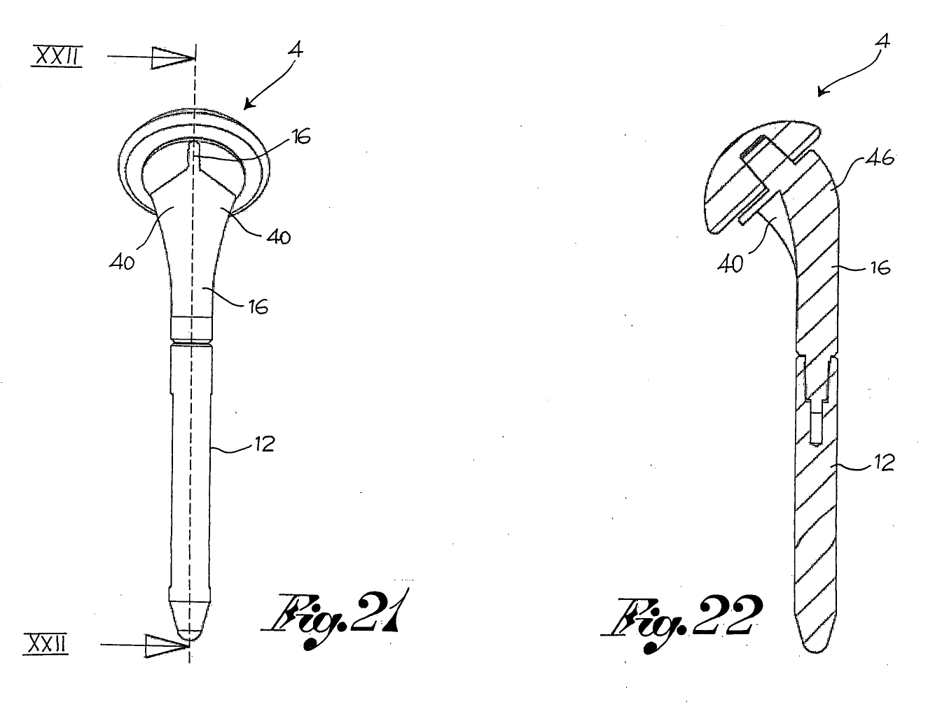

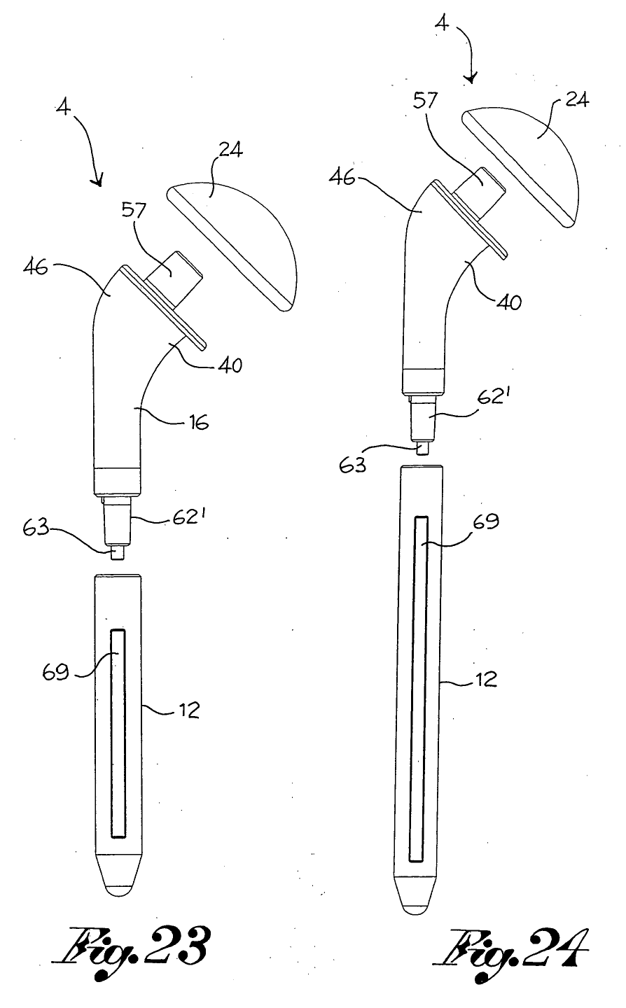

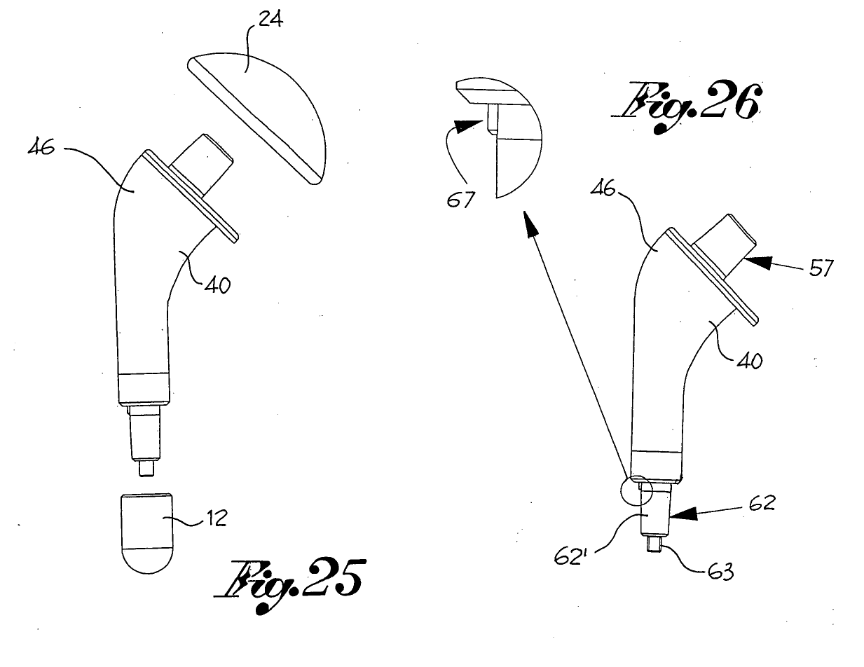

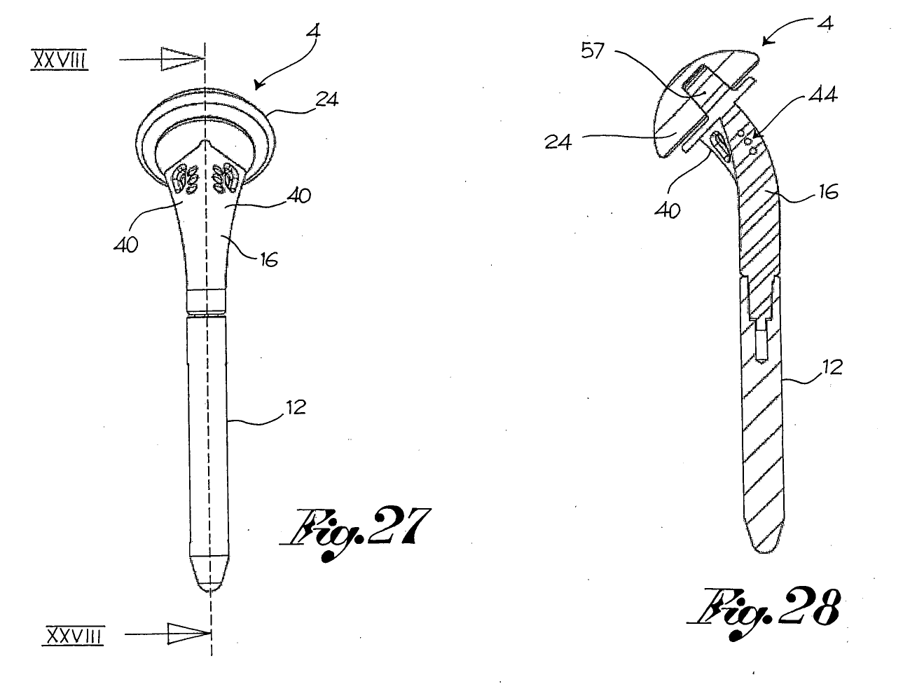

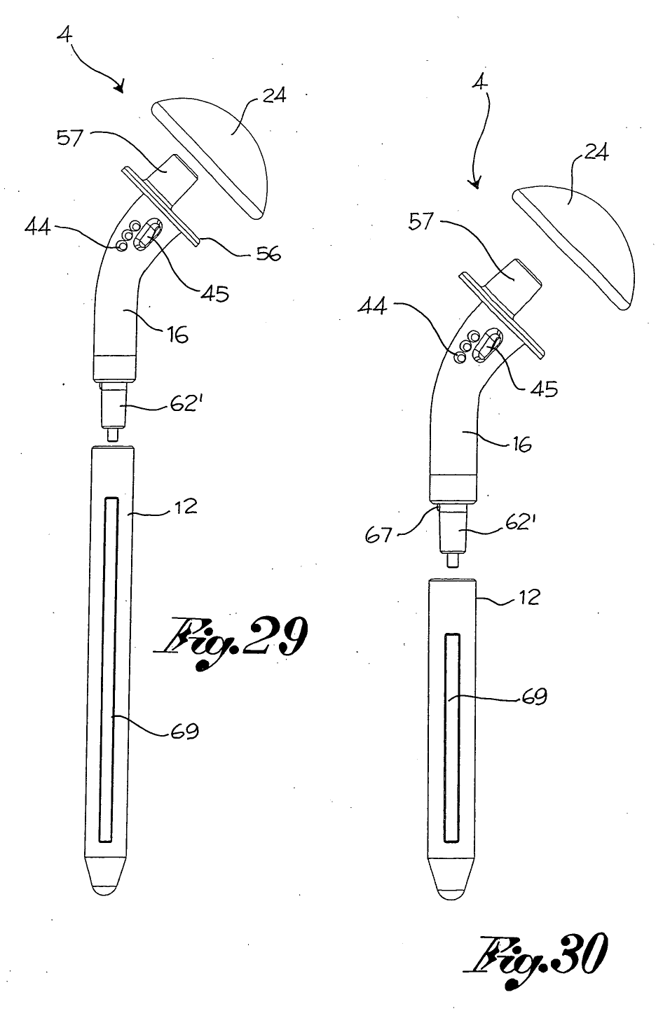

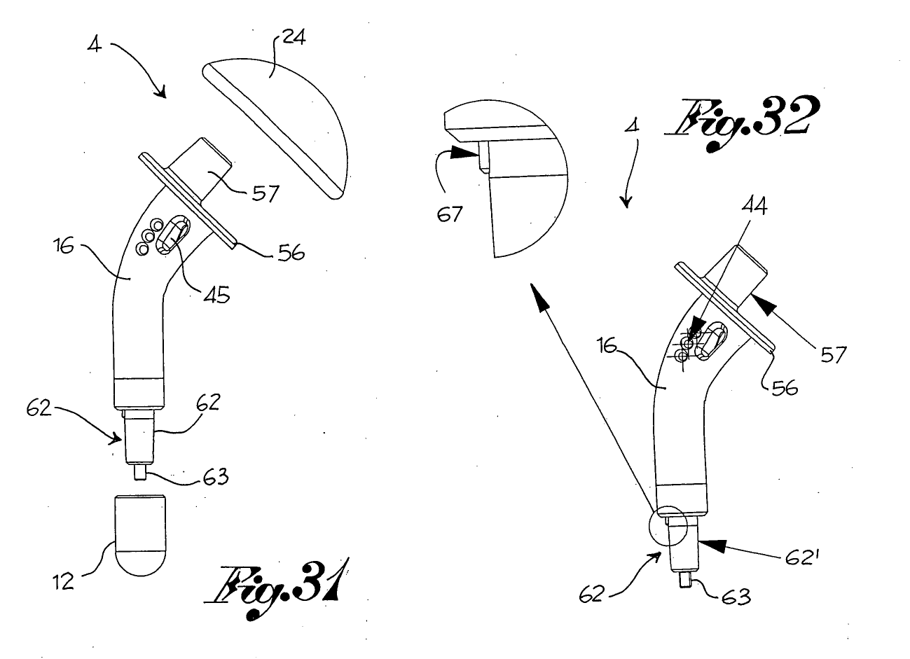

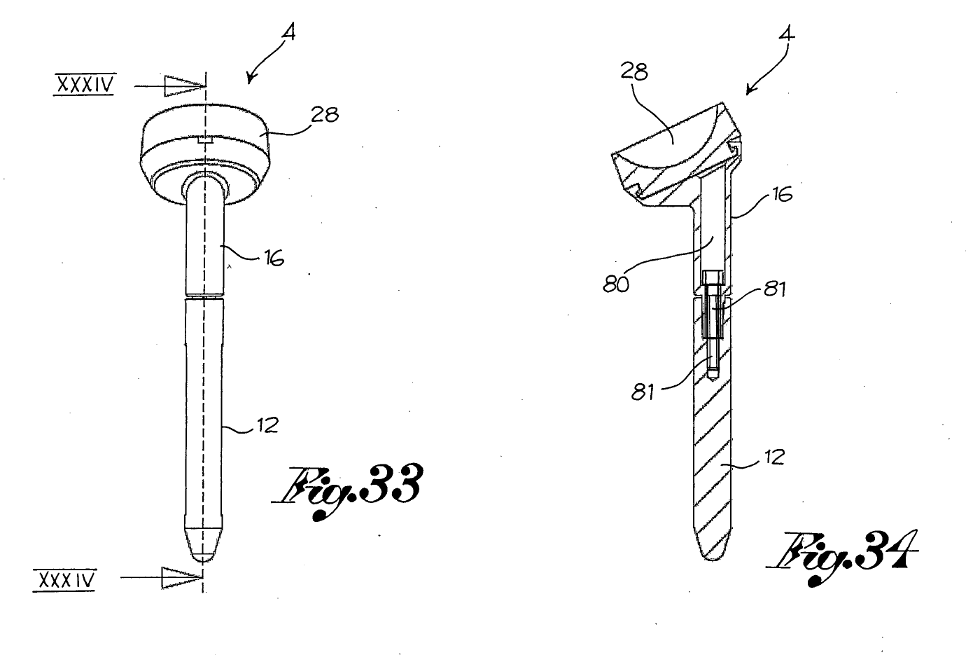

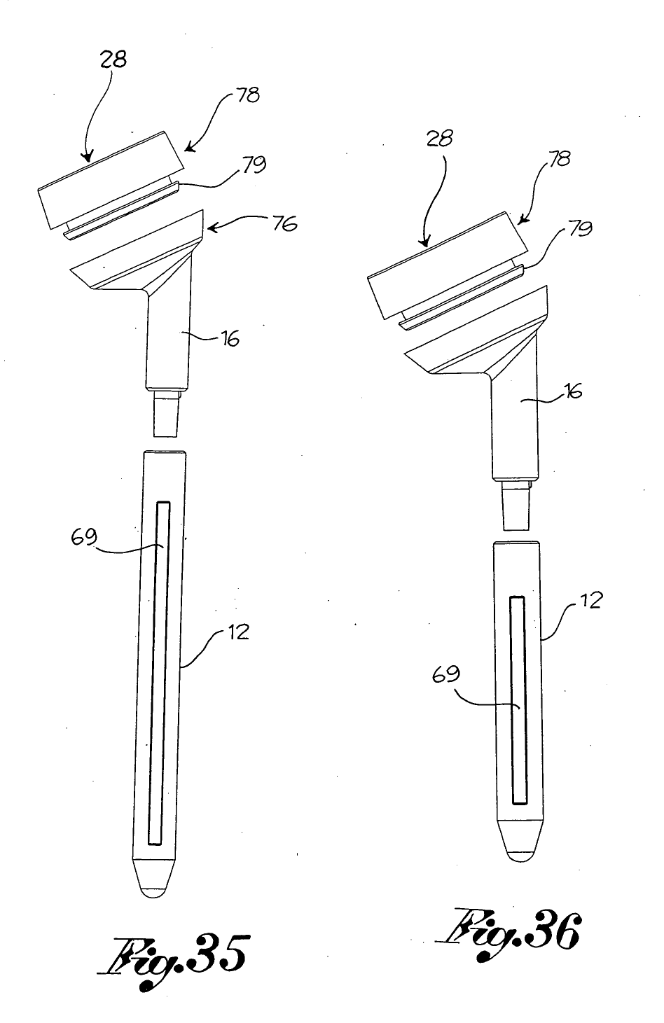

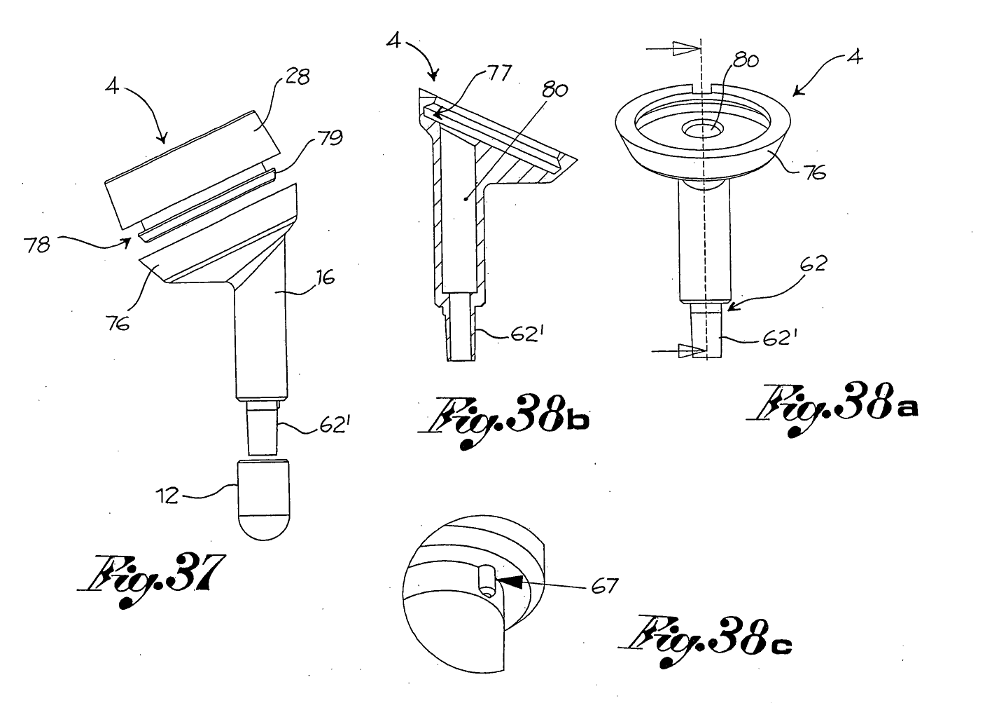

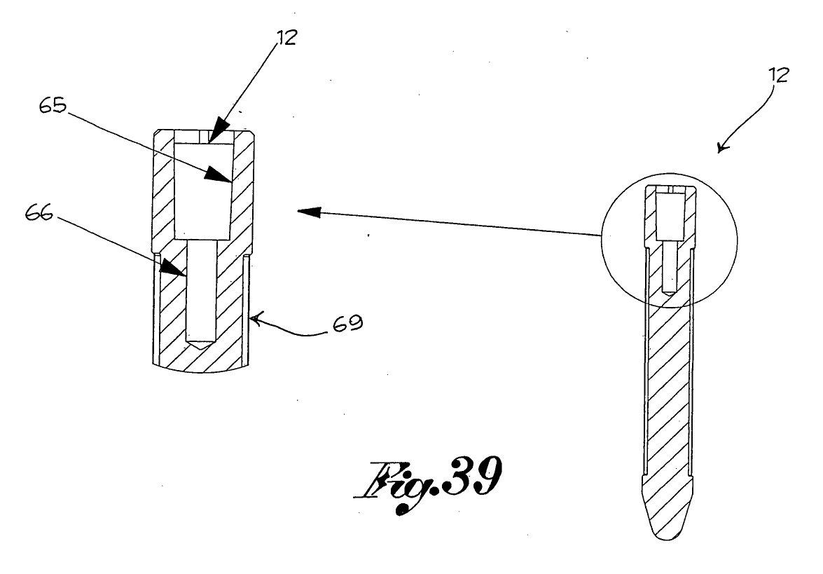

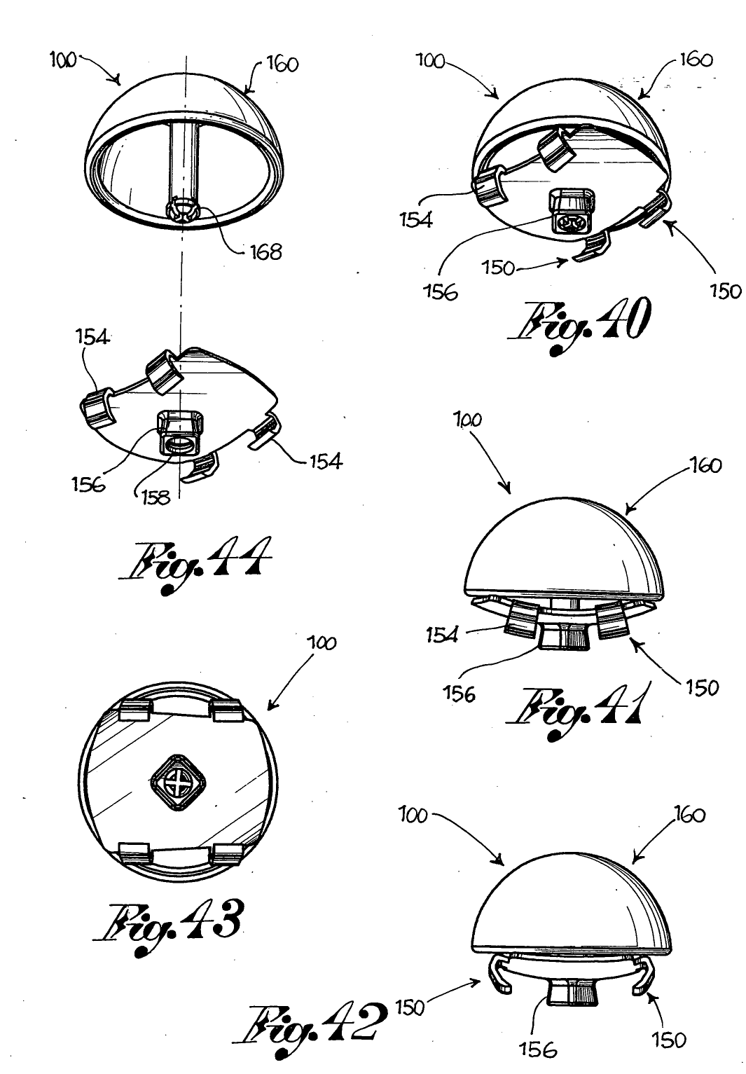

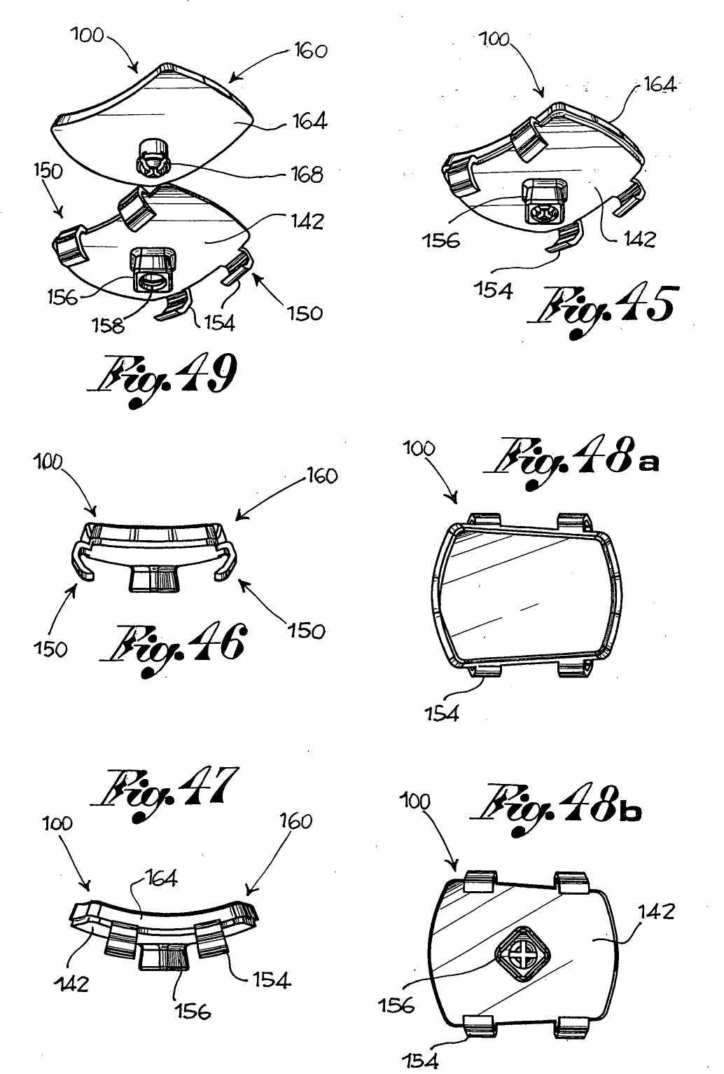

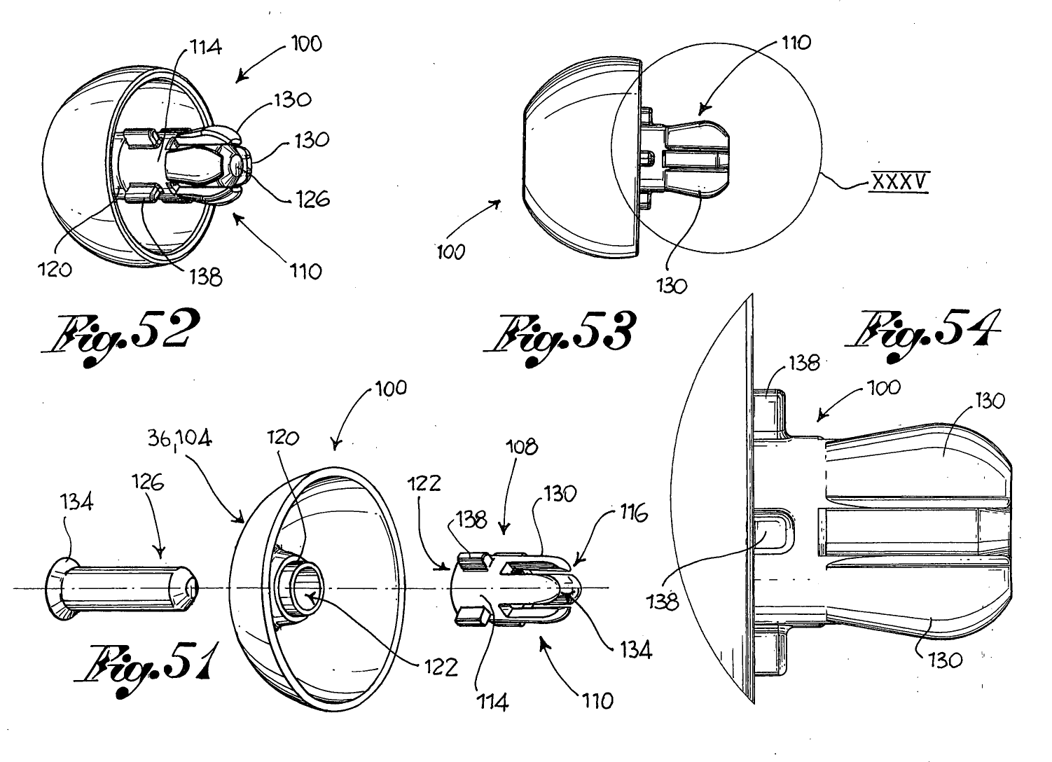

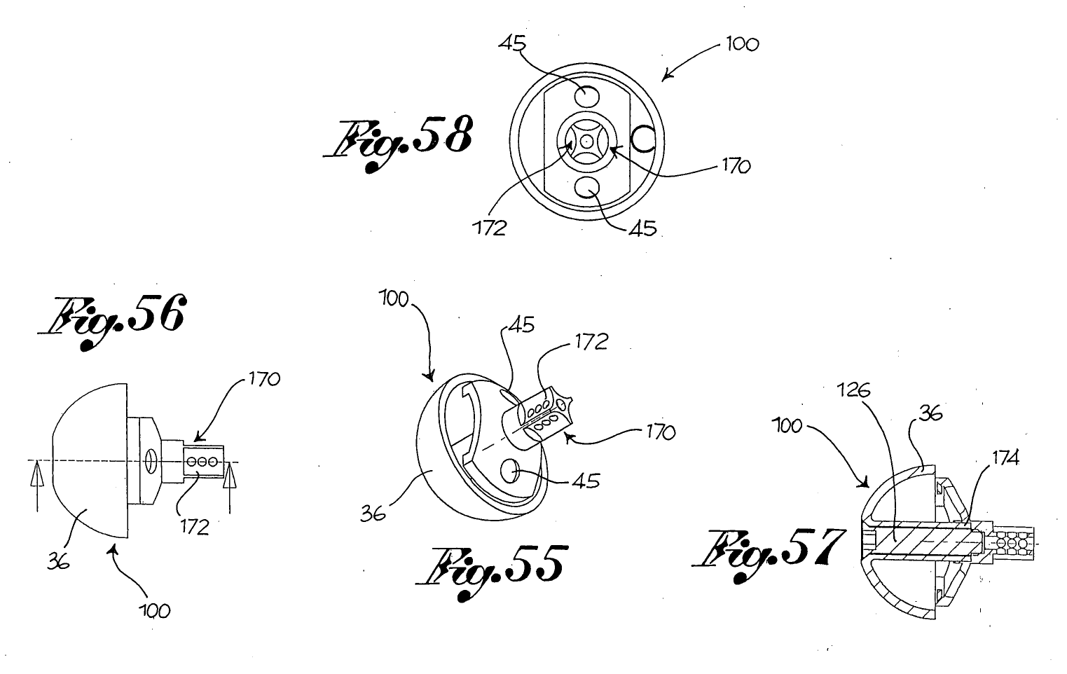

### DETAILED DESCRIPTION OF THE INVENTION

The common elements or parts of elements present in the embodiments described hereafter will be identified by the same numerals.

In reference to the aforesaid figures, according to one embodiment of the present invention throughout, the numeral 4 indicates a shoulder prosthesis suitable for insertion in a humerus 6, preferably positioned on the head 8 of said humerus 6.

According to one embodiment, the prosthesis 4 comprises a stem 12 suitable for insertion into the humerus to attach the prosthesis 4 on the humerus 6, the stem identifying a direction of insertion X.

The prosthesis 4, according to one embodiment, comprises a main body of the prosthesis 16 or metaphysary component, attachable to said stem 12, and adapted for insertion at least partially into the humerus positioned on the humeral head 8 having a pair of tuberosities 20.

Preferably, the stem 12 has an axis of symmetry in relation to the insertion direction X, in a manner to form a revolving surface adapted to rotation inside said humerus to permit the correct angular positioning of the prosthesis 4 in the humerus.

The main body of the prosthesis 16 extends according to a prevalent extension Y set at an angle in relation to the insertion direction X in a manner to follow the anatomical angle of the humeral head 8. Preferably said prevalent extension Y forms a curved line.

The main body of the prosthesis 16 is adapted to being associated with a spherical head 24 or with a cup 28 in a manner to interact with a glenoid cavity 32, if necessary covered with a relative plate 142, or with a glenosphere 36, respectively.

Advantageously, the main body of the prosthesis 16 comprises at least two support fins 40 that together form, in relation to a plane perpendicular to said prevalent extension Y, a flare angle a between 60 and 120 degrees in a manner to be abutted by said tuberosities 20 of the humerus, by the internal part of the bone, in an insertion configuration of the prosthesis in the humerus.

Preferably, said fins 40 together form, in relation to a plane perpendicular to said set at an angle direction, a flare angle a between 80 and 100 degrees.

Even more preferably, said fins 40 together form, in relation to a plane perpendicular to said set at an angle direction, a flare angle a equal to 90 degrees.

Advantageously, the support fins 40 present at least one hole 44 adapted to allowing the passage of a suture for the resuturing of said tuberosities 20.

Preferably, said fins 40 present three holes 44 arranged along the prevalent extension Y of the main body of the prosthesis 12, said holes 44 being adapted to allowing the passage of a suture for the resuturing of said tuberosities 20.

According to a possible embodiment, said fins 40 present slots 45 adapted to allowing an easier passage of the suture for the resuturing of said tuberosities 20 as well as to permit the insertion of the bone fragments to favour the osteointegration of the prosthesis 4.

Advantageously, said support fins 40 have a blade-like configuration and extend from said prevalent extension Y, in such a manner that the main body of the prosthesis 16 assumes a general section that is at least bilobate.

According to one embodiment, the main body of the prosthesis 16 comprises at least a third fin 46, preferably positioned symmetrically in relation to said support fins 44, said third fin extending from the opposite side to the support fins 44 in relation to the prevalent extension Y, in such a manner that the main body of the prosthesis 16 assumes a general “Y” shaped cross-section.

According to one embodiment, at least one of said fins 40, 44 extends, in relation to the axis of the prevalent extension Y, in an increasing distance, moving from a first end 50 of the main body of the prosthesis 16, from the attachable stem 12, to a second end 52 of the main body of the prosthesis 16, opposite to said first end 50.

Preferably, the configuration of the main body of the prosthesis 16 comprising two fins 40 and a third fin 46 is adapted to being used for shoulder arthrosis pathologies; a configuration comprising two fins 40, without the third fin 46, is preferably used in the case of fractures of the humeral head 8.

According to one embodiment of the present invention, said fins 40,44 opposite the stem 12, are operatively connected to a plate or disc 56 attachable by means of form coupling to a coupling portion 60 adapted to creating kinematic coupling with the glenoid.

According to a further embodiment of the present invention, said disc 56 is associated with a first cone 57, preferably of the ‘morse’ type adapted to favouring the fixing action of said coupling portion 60 to the main body of the prosthesis 16, by means of form coupling. The coupling portion 60, in the form of a spherical head 24 for example, will be provided with a housing having the counter form of the first cone 57 in a manner to ensure the secure blocking of the spherical head 24 on the main body of the prosthesis 16.

Preferably, the connection between the main body of the prosthesis 16 and the stem 12, positioned at said first end 50 of the main body of the prosthesis 16, is achieved by means of form coupling.

For example, the stem 12 comprises a threaded pin, which is screwed directly into a relative nut screw 61 of the main body of the prosthesis 16. Preferably, the main body of the prosthesis 16, positioned at said first end 50, comprises a connecting protuberance 62, presenting a second cone 62′, preferably of the ‘morse’ type, and a threaded portion 63. The stem 12 possesses a conical seat 65 adapted to blocking inside the second cone 62′ and a threaded hole 66. According to a possible embodiment, said threaded hole 66 is adapted to being screwed into the threaded portion 63 of the connecting protuberance 62.

Preferably, the connecting protuberance 62 comprises a rotation-proof ridge 67 adapted to preventing undesirable rotation between the stem 12 and the main body of the prosthesis 16.

According to a possible embodiment, the stem 12 presents at least one groove 69, being parallel to the direction of the prevalent extension X-X for example, in a manner to create a rotation proof action of stem 12 following the new growth of the bone; in other words, as the bone grows it will penetrate the groove 69 to form an undercut.

According to one embodiment, the coupling portion 60 comprises a spherical head 24, on the opposite side of the main body of the prosthesis 16, adapted to creating kinematic coupling with a glenoid 64 of the attachable scapula.

According to one embodiment, said disc 56 presents at least one ribbing 68 positioned on an axial rim and said coupling portion presents at least one groove 72 having the counter form of said ribbing 68.

Preferably, the disc 56 comprises four ribbings 68 in step formation, preferably perpendicular to each other, and in turn, the coupling portion 60 presents four grooves 72 set perpendicular to one another to permit coupling with said ribbing 68.

Preferably, the area of the section of the fins 40,44 in relation to a plane perpendicular to said prevalent extension Y does not exceed 85% of the total area of a disc associated with the main body of the prosthesis for the connection of the main body of the prosthesis to a coupling portion 60.

According to a further embodiment of the present invention, said coupling portion 60 comprises a cup 28 adapted to create kinematic coupling with a glenosphere 36 associated with the corresponding scapula.

According to an advantageous embodiment of the present invention, the main body of the prosthesis 16 is associated, by means of a male-female type form coupling to a coupling portion 60 in the form of a cup 28 adapted to interacting with a glenosphere 36 attachable to a corresponding glenoid 64.

According to one embodiment, the main body of the prosthesis 16 comprises a first connecting element 76, for example parallel to said prevalent extension Y, and the coupling portion 60 comprises a second connecting element 78 having the counter form of said first connecting element 76.

Advantageously, the coupling between the main body of the prosthesis 16 and the cup 28 is an axial type according to a coupling direction parallel to said first and second connecting element 76,78 in a manner to permit the fastening and release of the cup 28 from the main body of the prosthesis 16 in a same axial coupling direction, preferably parallel to said prevalent extension Y.

Advantageously, in an insertion configuration of the cup 28 into the main body of the prosthesis 16, a lower portion 82 of the cup 28, facing the main body of the prosthesis 16, results as aligned with a lower base 84 of said first connecting element 76, said lower base 84 being in a position facing the main body of the prosthesis 16.

According to one embodiment of the present invention, the first connecting element 76 presents a disc 56 having on its side surface, at least one ribbing 68 and said second connecting element 78 presents at least one groove 72 having the counter shape of said ribbing 68.

According to one embodiment of the present invention, said disc 56 presents four ribbings 68 positioned perpendicular to each other and said second connecting element 78 presents four grooves positioned perpendicular to each other to permit coupling with the ribbings.

According to one embodiment, the cup 28 comprises a wedge-shaped portion 88 in order to direct the cup 28 in a direction at an incidence with the glenosphere 36 of the attachable scapula.

Preferably, the area of the section main body of the prosthesis 16 in relation to a plane perpendicular to said prevalent extension Y does not exceed 85% of the total area of a disc 56 associated with the main body of the prosthesis 16 for the connection of the main body of the prosthesis 16 to a coupling portion 60, such as a cup 28.

According to a further embodiment, the first connecting element 76, for example set parallel to said prevalent extension Y, comprises a pocket 77 and the coupling portion 60, in the form of a cup 28, comprises a second connecting element 78 having the counter form of said first connecting element 76. For example, the second connecting element 78 is equipped with an annular ridge 79 suitable for insertion into said pocket 77 by means of form coupling.

Preferably, the main body of the prosthesis 16 is made of a metallic material and the cup 28 is made of a polymer material, preferably very high-density polyethylene (UHDPE). Alternatively, the cup 28 can be made of metallic material. Advantageously, the cup 28 presents a pass-through hole 80, which crosses the body 16 completely in a manner to permit the insertion of a connecting screw 81 between the main body of the prosthesis 16 and the stem 12. The connecting screw 81 presents a threaded section adapted to engagement with the threaded hole 66 of the stem 12. This makes it possible to change from the direct configuration to the inverted configuration of the prosthesis 4 by extracting the main body of the prosthesis 16, previously inserted in the humerus and mounted with a spherical head 24 according to the direct configuration concept, and by inserting a different main body of the prosthesis 16 mounted with a cup 28. Said main body of the prosthesis 16 is preferably circular and free of any fins 40,46.

In particular, the main body of the prosthesis 28 mounted with a cup can be anchored to the stem 12 which has remained inserted into the humerus by means of the insertion of the connecting screw. The removal of the main body of the prosthesis 16 from the stem is made easier by the connection between the main body of the prosthesis 16 and the stem 12 by means of a “morse” cone coupling. After the insertion of the main body of the prosthesis in the inverted configuration, in other words, equipped with a first connecting element 76 having a pocket 77, and after the screwing of the relative connecting screw, it is possible to fasten the cup by means of a click-on action to the main body of the prosthesis by fastening the annular ridge 79 in the pocket 77.

According to a further embodiment of the present invention, said prosthesis 4 comprises means of correction 92 for the relative position between the main body of the prosthesis 16 and the coupling portion 60, adapted to allowing a relative axial sliding action between the disc and the coupling portion 60 along at least one axial direction belonging to a plane perpendicular to said prevalent extension.

According to one embodiment, the means of correction comprises a first distancer 94 adapted to creating a form coupling with said disc 56, and a second distancer 96 adapted to creating a form coupling with the coupling portion 60, said first and second distancers 94,96 being able to move along at least one axial direction belonging to a plane perpendicular to said prevalent extension Y of the main body of the prosthesis 16.

According to one embodiment, at least one of said first and second distancers 94,96 comprises a slide shoe 98 adapted to allowing relative sliding action between said distancers.

The distancers 94,96 can be reciprocally blocked with each other along the slide by means of a continuous type or a discrete type regulation.

According to a further embodiment, the present invention relates to a shoulder prosthesis 100 adapted to being associated with a glenoid 64. The shoulder prosthesis 100 comprises a coupling element 104 suitable for attachment to a glenoid 64 to create a kinematic coupling with an attachable humerus and fixing means 108 of the coupling element 104 to the glenoid 64.

According to one embodiment, the fixing means 108 comprises an expanding element 110 suitable for insertion into a bone in a non-expanded configuration inside the bone in order to provide at least an undercut adapted to preventing the exit of the coupling element 104 from the bone.

The expanding element 110 comprises a portion for insertion 114, suitable for association with the coupling element 104, on the opposite side of the glenoid, and an expanding portion 116.

Preferably, the portion for insertion 114 forms an insertion coupling with the collar 120 of said coupling element 104.

The collar 120 and the portion for insertion 114 form a pass-through cavity 122.

Preferably, the prosthesis 100 comprises a pin 126 suitable for insertion into said cavity 122 through the collar 120 and the portion for insertion 114, in a manner so that it penetrates the expanding portion 116 and provokes expansion.

The expanding portion 116 comprises at least one tang 130 adapted to bending during the change from a non-expanded configuration to an expanded configuration.

Preferably, the expanding portion 116 comprises a plurality of tangs 130 that radially encircle a lower opening 134 in relation to the cavity 122 or in any case, to the diameter of the pin 126 in such a manner that after the insertion of a pin 126, this will interact with said tangs 130 provoking the expansion towards the exterior.

The pin 126 presents a beat head 134 adapted to acting as the end of run during the pin 126 insertion on the coupling element from the opposite side of the expanding element.

Preferably, the portion for insertion 114 comprises ledges 138 adapted to stabilising the prosthesis during rotation and flexion. Preferably, said ledges are positioned along an external rim of said collar.

According to one embodiment, the coupling element 104 is a glenosphere 36 adapted to being fixed to a scapula in such a manner that it will couple with a cup 28 operatively connected to a humerus.

According to a further embodiment, the coupling element 104 is a glenoid prosthesis being concave on the side of the attachable humerus and adapted to being associated with a head of a humerus.

According to a further embodiment, the shoulder prosthesis 100, adapted to being associated with a glenoid, comprises a plate 142 adapted to being fixed to a glenoid 64 to create a kinematic coupling with a head of a humerus. The prosthesis 100 comprises a plate element 146 and means for fastening to the glenoid 150. Preferably, the fastening means 150 comprises at least a catch 154 adapted to fastening onto a rim of the glenoid.

Preferably, the plate element 146 comprises at least two catches positioned on the opposite sides of the plate element, and even more preferably comprises two pairs of catches positioned on the opposite sides of the plate element.

According to one embodiment, the plate element 146 comprises a protuberance 156 adapted to being inserted at least partially into the glenoid 64.

The plate element 146 presents a coupling hole 158 adapted to allowing the insertion onto the plate element 146 of a coupling interface 160 to a humerus.

For example, said coupling interface 160 can be a coating plate 164 adapted to covering the plate element 146 on the opposite side from the glenoid, said plate 164 being concave on the side of the attachable humerus.

The coating plate 164 presents a ridge 168 adapted to being inserted and fixed in said coupling hole 158 of the plate element 146.

According to a further embodiment, said coupling interface 160 is a glenosphere 36 that is convex on the side of the attachable humerus.

According to a further embodiment of the present invention, the shoulder prosthesis 100 comprises a coupling element 104 in the form of a glenosphere 36, associated with the plate 142 adapted to being fixed to a glenoid 64 to create a kinematic coupling with a head of humerus. The plate 142 is produced in a single piece with an attachment protuberance 170 adapted to being inserted into the glenoid 64, preferably having at least a flared or bevelled form 172 to prevent relative rotation between the coupling element 104 and the attachable glenoid 64. Preferably, the fixing protuberance 170 comprises four flared elements 172; preferably at least one flared element having a pass-through hole adapted to favouring the osteointegration of the prosthesis.

Preferably, the prosthesis comprises said cavity 122 adapted to housing the pin 126 to fasten the glenosphere 36 to plate 142 and to the attaching protuberance 170. The plate 142 preferably presents holes or slots 45 for the possible insertion of fixing pins or to facilitate osteointegration.

As can be appreciated from the aforesaid description, the prosthesis of the present invention is able to overcome all the drawbacks present in prostheses of prior art.

In particular, the prosthesis according to the invention is especially non invasive, because of the reduced volume of the main body of the prosthesis.

The prosthesis according to the present invention provides far easier and optimal results in operations for resuturing the tuberosities of the humeral head (trochite and trochine) with the consequential improvement in articular function recovery. In fact the fins of the main body of the prosthesis create an advantageous support for the tuberosities during the resuturing stage, and as well as making resuturing operations easier for the surgeon, they also ensure correct support for the bone.

The prosthesis according to the present invention allows to correct certain important geometrical parameters such as anterior and posterior and central-side offset in the head-acromion distance without the need for explanting the prosthesis in question.

The prosthesis according to the present invention provides the possibility of post-operatory correction following error or for corrective purposes.

Thanks to the fact that the connecting section of the cup to the main body of the prosthesis is positioned above the anatomic neck of the humerus, this limits further invasive interventions of the prosthesis during any change from direct configuration to inverted configuration.

The prostheses according to the present invention provide safe attachment to the bone without being particularly invasive, and especially in relation to the glenoid. In fact, the use of an expanding element allows a single hole to be realised thus resulting far less invasive compared to the plurality of holes of the screw systems used in prior art.

The drilling of a single hole also limits the danger of drilling errors or ill-positioned holes, as well as the risk of bone fracture in proximity to the hole.

The anchoring system of the prosthesis to the glenoid by means of click-in fins provides the possibility of avoiding hole drilling in the bone; this provides a non-invasive method and yet guarantees very high stability. The elasticity of the fins also permits the correction of positioning errors or in any case, the removal of the prosthesis during implanting without the need for drilling.

The use of a stem without any ribbing permits the humerus to be inserted without damaging the humerus spongy tissue and furthermore, also allows any correction of the angular orientation of the stem and of the main body of the prosthesis that may be necessary without damaging the spongy tissue.

Therefore the use of a stem without ribbing contributes towards limiting invasive intervention of the prosthesis.

Advantageously, the area of the sections of the main body of the prosthesis, such as the fins for example, does not exceed 85% of the total area of the connecting element of the main body of the prosthesis to the coupling portion. In this manner the connecting element, having the form of a disc for example, resists against the penetration of the prosthesis into the humerus to ensure that the connecting element always remains in a position that is not lower than the neck of the humeral head.

In order to satisfy pertinent and specific necessities, those skilled in the art will be able to apply numerous modifications and variants to the aforesaid prosthesis, while remaining within the scope of the invention as defined in the following claims.

1-74. (canceled)

75. Shoulder prosthesis comprising
a stem suitable for being inserted into a humerus for the attachment of the prosthesis on the humerus, the stem forming a direction of insertion
a main body of the prosthesis, attachable to said stem, and suitable for being inserted at least partially into a humerus positioned on a humeral head presenting a pair of tuberosities
the main body of the prosthesis extending according to a principal axis set at an angle in relation to the insertion direction in a such a manner to follow the anatomic angle of the humeral head,
the main body of the prosthesis being adapted to be associated with a spherical head or with a cup in a manner so that it interacts with a glenoid cavity or with a glenosphere respectively, wherein
the main body of the prosthesis comprises at least two support fins that together form, in relation to a plane perpendicular to said principal axis, a flare angle between 60 and 120 degrees in a manner to be abutted by the tuberosities of the humerus in an insertion configuration of the prosthesis in the humerus.

76. Shoulder prosthesis according to claim 75, wherein said flare angle is between 80 and 100 degrees.

77. Shoulder prosthesis according to claim 75, wherein said flare angle is 90 degrees.

78. Shoulder prosthesis according to claim 75, wherein said fins have at least one hole or slot adapted to allowing the passage of the suture for the resuturing of said tuberosities.

79. Shoulder prosthesis according to claim 78, wherein said fins have three holes arranged along the principal axis of the main body of the prosthesis, said holes being adapted to allowing the passage of suture for the resuturing of said tuberosities.

80. Shoulder prosthesis according to claim 75, wherein said fins have a blade-like configuration and extend from said axis of principal axis, in a manner so that the main body of the prosthesis assumes an overall section having at least two lobes.

81. Shoulder prosthesis according to claim 75, wherein said main body of the prosthesis presents at least a third fin symmetrically arranged in relation to said support fins, said third fin extending from the side opposite the support fins in relation to the axis of principal axis in a manner so that the main body of the prosthesis assumes an overall ‘Y’-shaped section.

82. Shoulder prosthesis according to claim 75, wherein at least one of said fins extends in relation to the axis of principal axis for an increasing distance moving from a first end of the main body of the prosthesis, on the side of the attachable stem, to a second end of the main body of the prosthesis, opposite to said first end.

83. Shoulder prosthesis according to claim 75, wherein said fins, on the side opposite the stem, are operatively connected to a detachable disc by means of form coupling to a coupling portion adapted to creating a kinematic coupling with the scapula.

84. Shoulder prosthesis according to claim 83, wherein said coupling portion comprises a spherical head, on the opposite of the main body of the prosthesis, adapted to creating a kinematic coupling with a glenoid of the attachable scapula.

85. Shoulder prosthesis according to claim 83, wherein said disc presents at least one rib positioned on a shim and said coupling portion presents at least a groove having a shape complementary to that of said ribbing.

86. Shoulder prosthesis according to claim 84, wherein said disc has four ribs arranged at right angles to each other and said coupling portion presents four grooves arranged at right angles to each other in a manner to permit coupling with said ribs.

87. Shoulder prosthesis according to claim 83, wherein said coupling portion comprises a cup adapted to creating kinematic coupling with a glenosphere associated with the corresponding scapula.

88. Shoulder prosthesis according to claim 75, wherein said stem has an axis of symmetry in relation to said insertion direction, in a manner to provide a revolving surface adapted to rotation inside the said humerus to permit the correct angular positioning of the prosthesis therein.

89. Shoulder prosthesis according to claim 75, wherein the area of the fin section in relation to the plane perpendicular to said principal axis does not exceed 85% of the total area of the disc associated with the main body of the prosthesis for the connection of the main body of the prosthesis to a coupling portion.

90. Shoulder prosthesis according to claim 75, wherein the main body of the prosthesis comprises at a first end a first cone, of the Morse type adapted to favoring the attachment of a coupling portion of the prosthesis, such as a spherical head or a cup, to the main body of the prosthesis, by means of form coupling.

91. Shoulder prosthesis according to claim 90, wherein the coupling portion comprises a spherical head having a housing in the shape of the counter form of the first cone in a manner to ensure the secure blocking of the spherical head on the main body of the prosthesis.

92. Shoulder prosthesis according to claim 75, wherein the connection between the main body of the prosthesis and the stem, at one end of the main body of the prosthesis, is a form coupling.

93. Shoulder prosthesis according to claim 92, wherein the stem comprises a threaded pin that is screwed directly into a relative nut screw in the main body of the prosthesis.

94. Shoulder prosthesis according to claim 92, wherein the main body of the prosthesis, positioned at a first end, comprises a connecting protuberance, having a second ‘ morse’ type cone, and a threaded portion, and the stem having a conical seat adapted to blocking inside the second cone and a threaded hole.

95. Shoulder prosthesis according to claim 94, wherein said threaded hole is adapted to being screwed into the threaded portion of the connecting protuberance.

96. Shoulder prosthesis according to claim 94, wherein the connecting protuberance comprises a rotation-proof ridge adapted to preventing undesirable rotation between the stem and the main body of the prosthesis.

97. Shoulder prosthesis according to claim 75, wherein the stem presents at least a groove in a manner to prevent any rotation of the stem following bone regrowth.
